(19) United States
(12) Patent Application Publication
(10) Pub. No.: US 2019/0008631 A1
(43) Pub. Date: Jan. 10, 2019

(54) SYSTEMS AND METHODS WITH FENESTRATED GRAFT AND FILLING STRUCTURE

(71) Applicant: Endologix, Inc., Irvine, CA (US)

(72) Inventors: Aric Stone, Newport Beach, CA (US); James Machek, Clute, TX (US); Craig Welk, Tracy, CA (US); Mehdi Durali, Carlsbad, CA (US); Peter Nalbach, Mission Viejo, CA (US); Avyaya Sharma, San Diego, CA (US); Carlos Ortega, Irvine, CA (US); Girma Kebede, Lake Forest, CA (US); Ryan Goff, Irvine, CA (US); John Weaver, Aliso Viejo, CA (US); Swanand Sardesai, Anaheim Hills, CA (US); Sanaz Sotodeh, Irvine, CA (US); Jason Rinehart, San Marcos, CA (US)

(21) Appl. No.: 16/066,595

(22) PCT Filed: Dec. 23, 2016
(86) PCT No.: PCT/US16/68575

§ 371 Date: Jun. 27, 2018

Related U.S. Application Data
(60) Provisional application No. 62/274,093, filed on Dec. 31, 2015.

(51) Int. Cl. A61F 2/07; A61F 2/848

(52) U.S. Cl. CPC A61F 2/07; A61F 2002/061; A61F 2/848

(57) ABSTRACT

A system includes a graft body and a filling structure. The graft body has a fenestration in a side surface through which a support structure is insertable. The filling structure has an internal volume that is tillable with a filling medium and is configured to have a conduit through the internal volume through which the support structure is insertable. The conduit in the filling structure is alignable with the fenestration in the graft body such that the support structure is insertable through both the conduit in the filling structure and the fenestration in the graft body.

### CROSS-REFERENCE TO RELATED PATENT APPLICATIONS

This application claims priority from U.S. Provisional Application No. 62/274,093, filed Dec. 31, 2015, which is incorporated herein by reference in its entirety.

### FIELD

Embodiments of the present invention relate generally to endoluminal vascular prostheses and methods of placing such prostheses, and, in one application, to endoluminal vascular prostheses for use in the treatment of blood vessels with branches.

### BACKGROUND

An abdominal aortic aneurysm is a sac caused by an abnormal dilation of the wall of the aorta, a major artery of the body, as it passes through the abdomen. The abdomen is that portion of the body which lies between the thorax and the pelvis. It contains a cavity, known as the abdominal cavity, separated by the diaphragm from the thoracic cavity and lined with a serous membrane, the peritoneum. The aorta is the main trunk, or artery, from which the systemic arterial system proceeds. It arises from the left ventricle of the heart, passes upward, bends over and passes down through the thorax and through the abdomen to about the level of the fourth lumbar vertebra, where it divides into the two common iliac arteries.

The aneurysm usually arises in the infrarenal portion of the diseased aorta, for example, below the kidneys. When left untreated, the aneurysm may eventually cause rupture of the sac with ensuing fatal hemorrhaging in a very short time. High mortality associated with the rupture led initially to transabdominal surgical repair of abdominal aortic aneurysms. Surgery involving the abdominal wall, however, is a major undertaking with associated high risks.

Recently, a significantly minimally invasive clinical approach to aneurysm repair, known as endovascular grafting, has been developed, involving the transluminal placement of a prosthetic arterial graft in the endoluminal position (within the lumen of the artery). By this method, the graft is attached to the internal surface of an arterial wall by means of attachment devices (expandable stents), such as one above the aneurysm and a second stent below the aneurysm.

In certain conditions, the diseased region of the blood vessels extends across branch vessels. The blood flow into these branch vessels is critical for the perfusion of the peripheral regions of the body and vital organs. Many arteries branch off the aorta. For example, the carotid arteries supply blood into the brain, the renal arteries supply blood into the kidneys, the superior mesenteric artery (“SMA”) supplies the pancreas, the hypogastric arteries to the reproductive organs, and the subclavian arteries supply blood to the arms. When the aorta is diseased, the branch vessels may also be affected. Thoracic aortic aneurysms may involve the subclavian and carotid arteries, abdominal aneurysms may involve the SMA, renal, and hypogastric arteries. Aortic dissections may involve all branch vessels mentioned above.

### SUMMARY OF THE DISCLOSURE

A system in accordance with an embodiment includes a graft body and a filling structure. The graft body has a fenestration in a side surface through which a support structure is insertable. The filling structure has an internal volume that is fillable with a filling medium and is configured to have a conduit through the internal volume through which the support structure is insertable. In various embodiments, the fenestration in the graft body is aligned with the conduit in the filling structure, such that the support structure is able to pass through both the fenestration in the graft body and the conduit in the filling structure.

### BRIEF DESCRIPTION OF THE DRAWINGS

FIG. 1 is a partial section view of a patient's vasculature illustrating an embodiment of an endoluminal prosthesis deployed in the desired position within the patient's vasculature.

FIG. 2 is a front perspective view of a schematic representation of the endoluminal prosthesis illustrated in FIG. 1.

FIG. 3 is a cross-sectional view of the embodiment of the endoluminal prosthesis deployed in the patient's anatomy, taken through line 3-3 in FIG. 1.

FIG. 4 is a schematic representation of an embodiment of an endoluminal prosthesis positioned within an artery with a deployment catheter.

FIG. 5 is a schematic representations of an embodiment of an endoluminal prosthesis deployed in a desired position within an artery proximate to target branch arteries.

FIG. 6 is a schematic representation of an embodiment of an endoluminal prosthesis with pre-cannulated guidewires positioned proximate to the target branch arteries.

FIG. 7 is a schematic representation of an embodiment of an endoluminal prosthesis with pre-curved angiographic catheters tracked over the pre-cannulated guidewire.

FIG. 8 is a schematic representation of an embodiment of an endoluminal prosthesis with stents being positioned within the target branch arteries.

FIG. 9 is a schematic representation of an embodiment of an endoluminal prosthesis with stents being expanded within the target branch arteries and a filling structure partially supported with an inflatable balloon.

FIG. 10 is a schematic representation of an embodiment of an endoluminal prosthesis with a filling tube for delivering a hardenable filling medium to the filling structure.

FIG. 11 is a schematic representation of an embodiment of an endoluminal prosthesis with a fully inflated filling structure.

FIG. 12 is a perspective view of another embodiment of an endoluminal prosthesis.

FIG. 13A is a cross-sectional view of an embodiment of an endoluminal prosthesis deployed in a patient's anatomy.

FIG. 13B is cross-sectional view of an embodiment of an endoluminal prosthesis deployed in a patient's anatomy.

FIG. 14 is a partial section view of a patient's vasculature illustrating another embodiment of an endoluminal prosthesis in the patient's anatomy including a filling structure with a multitude of conduits.

FIG. 15 is a perspective view of a schematic representation of filling structure with a multitude of conduits of the embodiment of FIG. 14.

FIG. 16 is a partial section view of another embodiment of the endoluminal prosthesis deployed in the patient's anatomy including a multitude of horizontal filling structures.

FIG. 17 is a perspective view of a support structure extending between two horizontal filling structures in an uninflated state.

FIG. 18 is a perspective view of a support structure extending between two horizontal filling structures in an inflated state.

FIG. 19 is a partial section view of a patient's vasculature illustrating another embodiment of an endoluminal prosthesis deployed in a desired position within the patient's vasculature.

FIG. 20 is a front perspective view of a schematic representation of the embodiment of the endoluminal prosthesis illustrated in FIG. 19.

FIG. 21 is a cross-sectional view of the embodiment of the endoluminal prosthesis deployed in the patient's anatomy, taken through line 21-21 in FIG. 19.

FIG. 22 is a schematic representation of an embodiment of an endoluminal prosthesis positioned within an artery with a deployment catheter.

FIG. 23 is a schematic representation of an embodiment of an endoluminal prosthesis deployed in a desired position within an artery proximate to target branch arteries.

FIG. 24 is a schematic representation of an embodiment of an endoluminal prosthesis with pre-cannulated guidewires positioned proximate to target branch arteries.

FIG. 25 is a schematic representation of an embodiment of an endoluminal prosthesis with pre-curved angiographic catheters tracked over the pre-cannulated guidewire.

FIG. 26 is a schematic representation of an embodiment of an endoluminal prosthesis with stents being positioned within target branch arteries.

FIG. 27 is a schematic representation of an embodiment of an endoluminal prosthesis with stents being expanded within target branch arteries and a filling structure partially supported with an inflatable balloon.

FIG. 28 is a schematic representation of an embodiment of an endoluminal prosthesis with a filling tube for delivering a hardenable filling medium to a filling structure.

FIG. 29 is a schematic representation of an embodiment of an endoluminal prosthesis with a fully inflated filling structure.

FIG. 30 is a partial section view of a patient's vasculature illustrating another embodiment of an endoluminal prosthesis in the patient's anatomy including a main graft body with multiple fenestrations.

### DETAILED DESCRIPTION

The following detailed description is now directed to certain embodiments of the disclosure. In this description, reference is made to the drawings wherein like parts are designated with like numerals throughout the description and the drawings.

Certain embodiments described herein are directed to systems, methods, and apparatuses to treat lesions, aneurysms, or other defects in the aorta, including, but not limited to, the thoracic, ascending, and abdominal aorta, to name a few. However, the systems, methods, and apparatuses may have application to other vessels or areas of the body, or to other fields, and such additional applications are intended to form a part of this disclosure. For example, it will be appreciated that the systems, methods, and apparatuses may have application to the treatment of blood vessels in animals. In short, the embodiments and/or aspects of the endoluminal prosthesis systems, methods, and apparatuses described herein can be applied to other parts of the body or may have other applications apart from the treatment of the thoracic, ascending, and abdominal aorta. And, while specific embodiments may be described herein with regard to particular portions of the aorta, it is to be understood that the embodiments described can be adapted for use in other portions of the aorta or other portions of the body and are not limited to the aortic portions described.

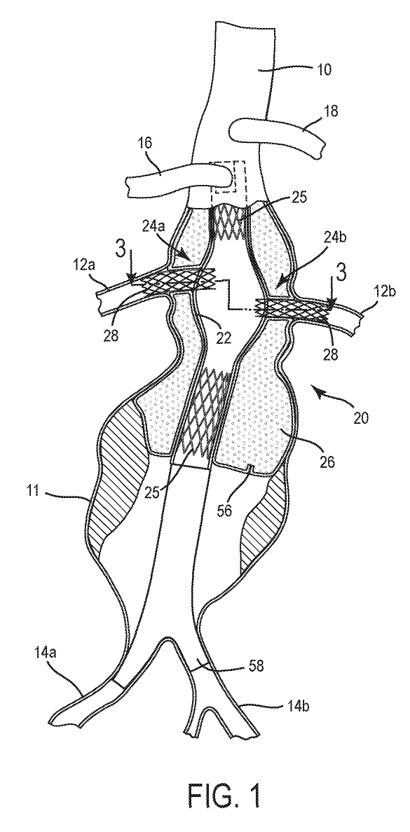

FIG. 1 is a partial section view of a patient's vasculature illustrating an embodiment of an endoluminal prosthesis deployed in a desired position within the patient's vasculature. Although the prostheses disclosed herein can be adapted for deployment in any suitable vessels in the body, some embodiments are described as being deployed in particular vessels or vascular regions within a patient's body. However, the particular prostheses illustrated are not limited to deployment in only one particular vessel or vascular region. In some embodiments, the embodiments shown can be adapted for deployment in other suitable vessels within a patient's body, including the aorta, thoracic artery, renal arteries, iliac arteries, etc.

As an example, with reference to FIG. 1, an embodiment of an endoluminal prosthesis 20 is shown deployed in a patient's aorta 10. For reference, also illustrated are a patient's first and second renal arteries 12a, 12b, respectively, a patient's first and second iliac arteries 14a, 14b, respectively, a patient's superior mesenteric artery (SMA) 16, and a patient's celiac artery 18. An infrarenal abdominal aortic aneurysm 11 is also shown between the renal arteries 12a and 12b and the iliac arteries 14a and 14b and may have regions of mural thrombus over portions of its inner surface.

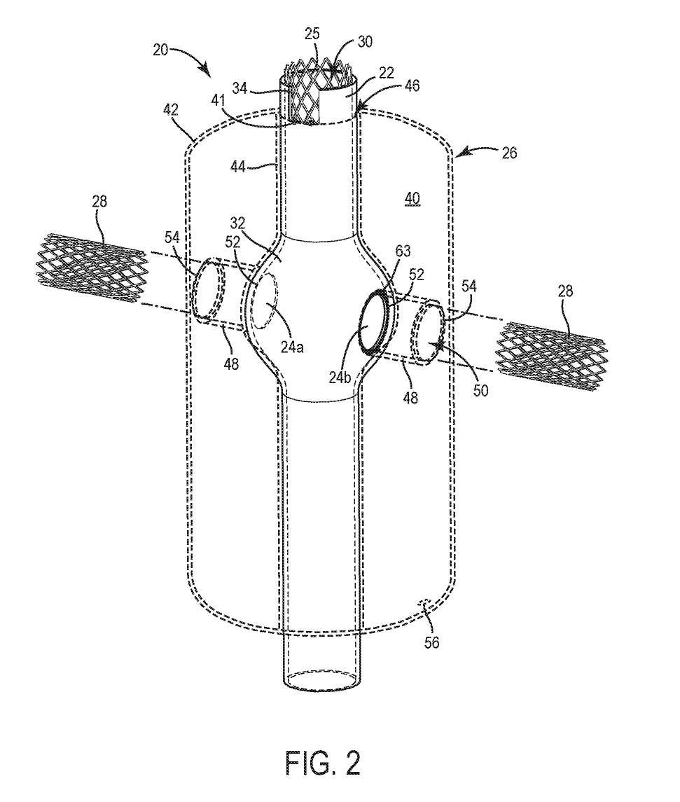

FIG. 2 is a front perspective view of a schematic representation of the endoluminal prosthesis 20 illustrated in FIG. 1. The embodiment of the endoluminal prosthesis 20 illustrated in FIGS. 1 and 2 includes a main graft body 22 including a first fenestration 24a (e.g., scallop, cutout, opening, etc.), and a second fenestration 24b, a support structure 25, a double-walled filling structure 26 in the area of the aneurysm 11 (shown in dashed lines in FIG. 2 for clarity), and support structures 28 extending through the fenestrations 24a, 24b and through the filling structure 26.

The endoluminal prosthesis 20 is described herein as being positioned in the abdominal aorta 10 proximate to one or more branch arteries with elements, such as the support structures 28 being positioned within the branch arteries. In some configurations, the elements can be positioned within any one or combination of the following: left renal artery, right renal artery, second lumbar, testicular, inferior mesenteric, middle sacral, or other vessels branching from the aorta. Thus, in some embodiments, the endoluminal prosthesis 20 can includes any number of elements that are required for the specific application, including, but not limited to, elements for one, two, three, or more branch arteries. Because the elements disposed in the branch arteries can be configured to conform to a wide range of vessels and a wide range of positions, the elements can be of any suitable size, shape, or configuration, and can be attached to the main graft body 22 in any of a wide variety of locations.

The main graft body 22 defines a central lumen 30. The main graft body 22 provides a synthetic vessel wall that channels the flow of blood through the diseased portion of the blood vessel (e.g., the aorta 10). The endoluminal prosthesis 20 is positioned with the first fenestration 24a and second fenestration 24b each aligned with a branch vessel of the aorta 10. In one embodiment, the first fenestration 24a is aligned with the first renal artery 12a and the second fenestration 24b is aligned with the second renal artery 12b such that a fluid path is formed from the central lumen 30 to the first renal artery 12a and second renal artery 12b. In some embodiments, the main graft body 22 can have a generally cylindrical, tubular shape. The endoluminal prosthesis 20 can be formed from any suitable material, such as, but not limited to, Polytetrafluoroethylene (PTFE), expanded PTFE (ePTFE), and paralyne.

Some embodiments of the endoluminal prosthesis 20 can be formed from an expandable material. The endoluminal prosthesis 20 can be formed such that the main graft body 22 can be larger than the target vessel into which the main graft body 22 is to be deployed. For example, the target can be the aorta 10, and the endoluminal prosthesis 20 can be deployed so as to span across the aneurysm 11 in the aorta 10. In some embodiments, the endoluminal prosthesis 20 may be positioned such that an enlarged portion 32 of the main graft body 22 is disposed proximate the first renal artery 12a and the second renal artery 12b. In various graft embodiments disclosed herein, the diameter of a graft body (such as, without limitation, the main graft body 22) or an enlarged portion of any embodiment of a graft body disclosed herein can be approximately 30% larger than a diameter of the target vessel or the diameter of the non-enlarged portion of the graft body. In some embodiments, the diameter of the graft body (such as without limitation the main graft body 22) or an enlarged portion of any embodiment of a graft body disclosed herein can be less than approximately 20% larger, or from approximately 20% to approximately 50% or more larger, or from approximately 25% to approximately 40% larger than the target vessel or the diameter of the non-enlarged portion of the graft body, or to or from any values within these ranges.

In some embodiments, the main graft body 22 can have any of the features of the main graft body disclosed in U.S. patent application Ser. No. 14/581,675, filed on Dec. 23, 2014 (titled “Fenestrated Prosthesis”), which is hereby incorporated by reference in its entirety as if fully set forth herein.

Further, in various of the graft embodiments disclosed herein, at least a portion of the graft material adjacent to the one or more fenestrations or openings can be free to translate in a circumferential or axial direction relative to the stent or other support structure that the graft is supported by. For example, without limitation, particular portions such as the end portions of the graft material can be sutured or otherwise fastened to the stent, while a mid portion of the graft having one or more fenestrations therethrough can be unattached to the stent so that such mid portion can be free to translate relative to the stent and, hence, permit the adjustability of the fenestrations relative to the stent. In this configuration, for example, the fenestrations can be adjusted to align with the ostium of the patient's branch vessels.

The oversized diameter of the main graft body 22 (e.g., the enlarged portion 32) can provide excess or slack graft material in the main graft body 22 such that the fenestrations 24a and 24b can each be moved in an axial or angular direction to align the fenestrations 24a and 24b with the branch vessels arteries, as will be described in greater detail below.

As described above, two or more fenestrations can be formed in the main graft body 22 at any desired location. For example, the two fenestrations 24a and 24b can be formed at generally diametrically opposed locations (refer to FIG. 2). However, any number of fenestrations can be formed in the main graft body 22 at any desired locations. Additionally, fenestrations can be formed in the distal end portion or at any suitable location in the main graft body 22, the scallops or cutouts being configured to prevent obstruction of other arteries branching off of the main vessel into which the main graft body 22 is to be deployed. For example, in some embodiments, an additional fenestration 34 can be formed in a distal portion of the main graft body 22. The fenestration 34 can be formed so as to align with the patient's SMA 16 and/or celiac artery 18.

In some embodiments, at least a portion of the main graft body 22 includes undulations, folds, bends, corrugations, or other similar features in the axial direction therein when the main graft body 22 is in a relaxed state, such as before the graft has been deployed. In some embodiments, a middle portion of the graft (e.g., the enlarged portion 32) includes undulations, folds, bends, corrugations or other similar features while the distal or upstream portion and/or the proximal or downstream portion define a smooth contour. In some embodiments, after the main graft body 22 has been deployed in the target vessel, because the main graft body 22 can have a larger diameter than the vessel diameter, folds, wrinkles, or other undulations (collectively referred to as folds) can form in the main graft body 22 about the circumference of the main graft body 22.

In some embodiments, the support structure 25 which can be, for example, a covered stent, a bare wire stent, etc., or any other suitable stent or anchoring device is deployed within the central lumen 30 of the main graft body 22 to secure the graft in the desired location. In various embodiments, the support structure 25 compresses the main graft body 22 against a wall of the vessel or against the filling structure 26 provided between the main graft body 22 and the wall of the vessel and secures the main graft body 22 and the fenestrations 24a and 24b in the desired locations. In various embodiments, the support structure 25 is formed from any number of resilient metals such as stainless steel, cobalt-chromium (CoCr), nitinol or resilient polymers. The support structure 25 may be coupled to the inside or outside surface of the main graft body 22. In some embodiments, the endoluminal prosthesis 20 includes a first section of the support structure 25 at a proximal portion of the main graft body 22 and a second section of the support structure 25 at a distal portion of the main graft body 22 such that the support structure 25 does not extend through the enlarged portion 32. In some embodiments, the support structure 25 extends through the enlarged portion 32. In various embodiments in which the support structure 25 is disposed in the enlarged portion 32, the fenestrations 24a, 24b extend through the support structure 25.

In various embodiments, the filling structure 26 surrounds the main graft body 22 and, when inflated, occupies the annular space between the main graft body 22 and the walls of the aorta 10. The filling structure 26 defines an internal volume 40 defined between an outer wall 42 and inner wall 44. The inner wall 44 defines an inner lumen 46. The inner lumen 46 is configured to receive the main graft body 22. In various embodiments, the geometry of the filling structure 26 is chosen or fabricated to match the particular patient geometry being treated. Upon inflation with a filling material or medium delivered into the internal volume 40, the outer wall 42 expands radially outwardly. In some embodiments, the filling structure 26 further includes at least one conduit 48 that extends through the internal volume 40 and is aligned with a fenestration of the main graft body 22. The embodiment illustrated in FIGS. 1 and 2 includes two conduits 48. Each conduit 48 defines a passage 50 that is separate from the internal volume 40. The passage 50 is open on a first end 52 into the inner lumen 46 and is open on a second end 54 into the space surrounding the filling structure 26. In some embodiments, the filling structure 26 is coupled to the main graft body 22 and/or the support structure 25 with sutures 41 to maintain a desired alignment of each conduit 48 with a corresponding fenestration, such as the fenestrations 24a, 24b in the main graft body 22. In some embodiments, the filling structure 26 is coupled to the main graft body 22 and/or the support structure 25 with an adhesive or other suitable attachment mechanism. In some embodiments, the filling structure 26 is integrally formed with the main graft body 22. For example, in some embodiments, the main graft body 22 forms an inner wall of the filling structure 26.

In various embodiments, the filling structure 26 includes at least one valve 56 to permit the introduction of the filling material or medium into the internal volume 40 of the filling structure 26. In some embodiments, the valve 56 includes a simple flap valve. In some embodiments, the valve 56 comprises other more complex ball valves, or other one-way valve structures. In some embodiments, the value comprises a two-way valve structure to permit both filling and selective emptying of the internal volume 40. In some embodiments, a filling tube includes a needle or other filling structure to pass through the valve 56 to permit both filling and removal of filling medium. In some embodiments, a radiopaque marker 63 is provided at each of the fenestrations, such as the fenestrations 24a, 24b, to facilitate locating the fenestrations.

In some embodiments, the filling structure 26 can have any of the features of the filling structures disclosed in U.S. patent application Ser. No. 13/285,897, filed on Oct. 31, 2011 (titled “Graft Systems Having Filling Structures Supported by Scaffolds and Methods of Their Use”), now U.S. Pat. No. 8,870,941, or any of the features of the filling structures disclosed in U.S. patent application Ser. No. 12/478,225, filed on Jun. 4, 2009 (titled “Sealing Apparatus and Methods of Use”), each of which are hereby incorporated by reference in their entirety as if fully set forth herein.

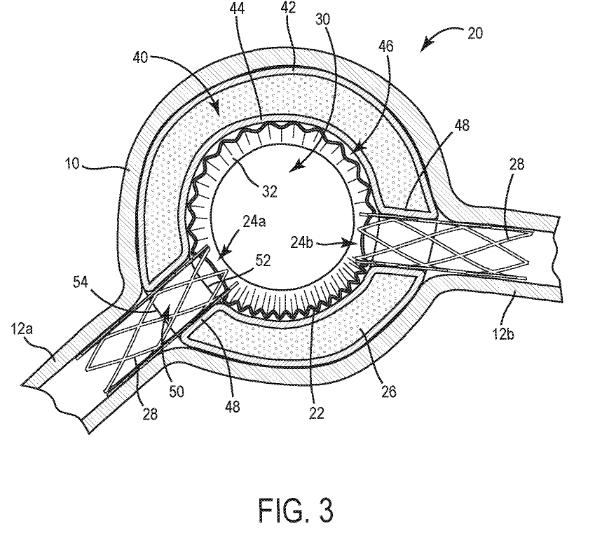

FIG. 3 is a cross-sectional view of the embodiment of the endoluminal prosthesis 20 deployed in the patient's anatomy, taken through line 3-3 in FIG. 1. Each support structure 28 (e.g., covered stent, bare wire stent, etc.) extends from the central lumen 30, through the corresponding fenestration 24a, 24b in the main graft body 22, through the corresponding conduit 48 of the filling structure 26, and into the corresponding renal artery 12a, 12b. In various embodiments, the support structures 28 are stent-like or graft-like vascular structures and are deployed within the tubular lumens using balloon or other expansion catheters (in the case of malleable or balloon-expandable scaffolds) or using constraining sheaths (in the case of self-expanding scaffolds). In various embodiments, the support structures 28 maintain open passages through the conduits 48 and prevent the conduits 48 from collapsing as the internal volume 40 is filled with the filling material or medium. In various embodiments, each support structure 28 is formed from any number of resilient metals such as stainless steel, cobalt-chromium (CoCr), nitinol or resilient polymers.

In various embodiments, the main graft body 22 and the filling structure 26 are positioned such that the fenestrations 24a, 24b and the conduits 48 are aligned with the ostia of the renal arteries 12a, 12b to provide a non-tortuous path for the support structures 28. In some embodiments, the main graft body 22 and the filling structure 26 are constructed individually based on the particular configuration of the blood vessels of a patient in which the endoluminal prosthesis 20 is placed. In some embodiments, the fenestrations 24a, 24b and/or the conduits 48 are oversized, such as having a cross-sectional height or width that is greater than the diameter of the support structure 28, to provide a greater flexibility in the placement of each support structure 28. The main graft body 22 and the filling structure 26 can be constructed in multiple configurations with different positioning of the fenestrations 24a, 24b and the conduits 48. In various embodiments, the main graft body 22 and the filling structure 26 are chosen to best matching the particular configuration of the blood vessels in which the endoluminal prosthesis 20 is to be placed.

With reference to FIGS. 1 and 3, in some embodiments, the endoluminal prosthesis 20 is deployed in the aorta 10 proximate the first renal artery 12a and second renal artery 12b. In some embodiments, the endoluminal prosthesis 20 is configured to interact with other prostheses. For example, in some embodiments, the endoluminal prosthesis 20 is configured to be deployed with a second endoluminal prosthesis 58 having a bifurcated graft for placement in the iliac arteries 14a, 14b. In some embodiments, the second endoluminal prosthesis 58 have any of the features of the endoluminal prosthesis disclosed in U.S. patent application Ser. No. 12/101,863, filed on Apr. 11, 2008 (titled “Bifrucated Graft Deployment System and Methods”), now U.S. Pat. No. 8,236,040, which is hereby incorporated by reference in its entirety as if fully set forth herein.

In various embodiments, the endoluminal prosthesis 20 is configured to mate with, interface with, or otherwise engage other prosthesis, such as the second endoluminal prosthesis 58 to provide a continuous conduit to facilitate blood flow through the diseased portion of the aorta 10. In some embodiments, a portion of the second endoluminal prosthesis 58 is received within the central lumen 30 of the main graft body 22. In some embodiments, a portion of the second endoluminal prosthesis 58 receives the main graft body 22 such that the portion of the second endoluminal prosthesis 58 is between the main graft body 22 and the filling structure 26. In various embodiments, the endoluminal prostheses 20, through the interconnection with the second endoluminal prosthesis 58 utilizes the anatomical bifurcation of the second endoluminal prosthesis 58 for fixation in addition to aneurysm sealing.

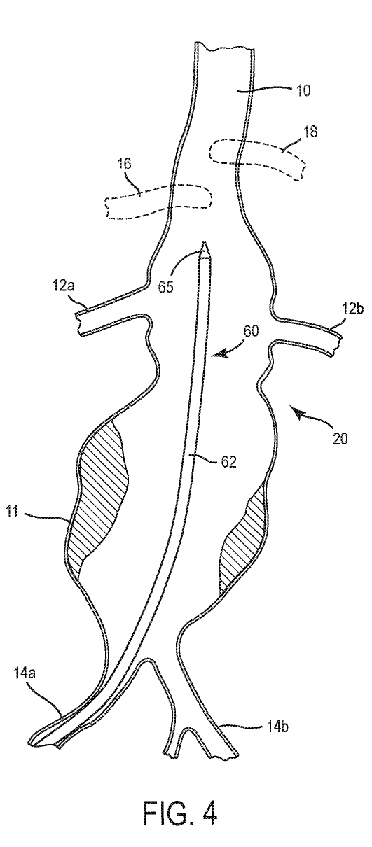

FIGS. 4, 5, 6, 7, 8, 9, 10, and 11 are schematic representations of a method in accordance with an embodiment of positioning and implanting the endoluminal prosthesis 20 in a desired aortic location. A deployment catheter 60 is positioned in the aorta 10, as illustrated in FIG. 4. The deployment catheter 60 includes a hollow outer sheath 62 in which the endoluminal prosthesis 20 is compressed and loaded for delivery to a desired location. According to an exemplary embodiment, the deployment catheter 60 is advanced over a guidewire through a puncture in the patient's groin accessing the iliac artery by the Seldinger technique. In various embodiments, the deployment catheter 60 is positioned to deploy the endoluminal prosthesis 20 in such a way as to maintain patency of the branch blood vessels. With reference to FIGS. 1, 2 and 4, in some embodiments the position and angular orientation (e.g., rotation) of the endoluminal prosthesis 20 is controlled such that each of the fenestrations 24a, 24b is aligned with a corresponding one of the renal arteries 14a, 14b (e.g., using the radiopaque marker 63 to facilitate positioning) and the fenestration 34 at the end of the main graft body 22 is aligned with the SMA 16.

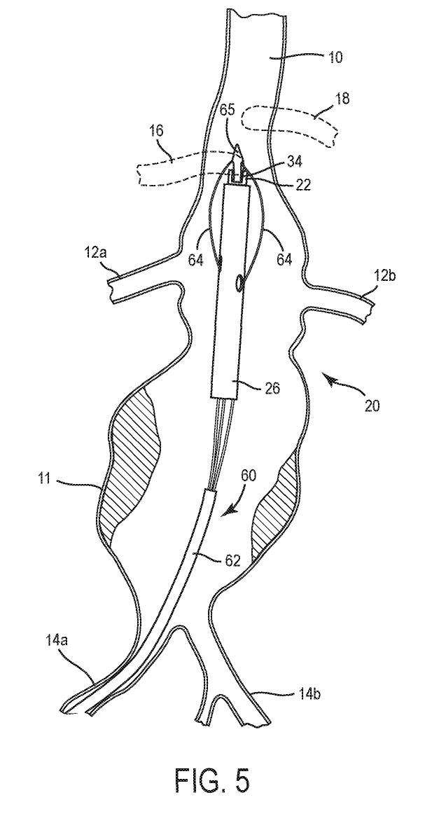

Once properly positioned within the aorta 10, the outer sheath 62 of the deployment catheter 60 is retracted to deploy the endoluminal prosthesis 20, as illustrated in FIG. 5. In some embodiments, the endoluminal prosthesis 20 is fixed in place during the procedure, such as with a lockwire. In some embodiments, at least one pre-cannulated guidewire 64 is provided. With reference to FIGS. 1, 2, and 5, in various embodiments, each pre-cannulated guidewire 64 extends from the central lumen 30 of the main graft body 22, through a corresponding fenestration 24a, 24b, through a corresponding conduit 48 in the filling structure 26, and out towards a nosecone 65 of the deployment catheter 60. In various embodiments, each pre-cannulated guidewire 64 runs under the support structures 28, which is stored in a collapsed state within the sheath 62. In some embodiments, one or more radiopaque markers 63 are provided at one or more of the fenestrations 24a, 24b to facilitate the positioning of each pre-cannulated guidewire 64. In some embodiments, additional radiopaque markers are provided on the outer wall 42 and/or the inner wall 44 of the filling structure 26 proximate the conduits 48 to further facilitate positioning of elements within the patient's body.

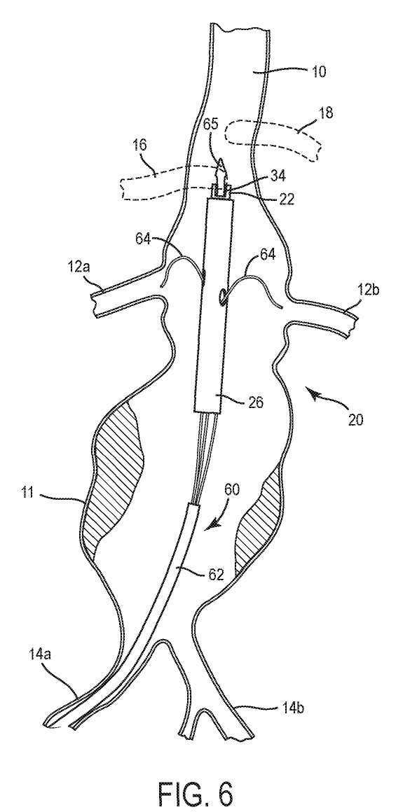

In some embodiments, two pre-cannulated guidewires 64 are provided in the approximate locations of the renal arteries 12a, 12b, as illustrated in FIG. 6. However, in other embodiments, three or more pre-cannulated guidewires are provided to accommodate additional vessels, such as the celiac artery 18 or the SMA 16 if the endoluminal prosthesis 20 is configured with support structures for additional vessels.

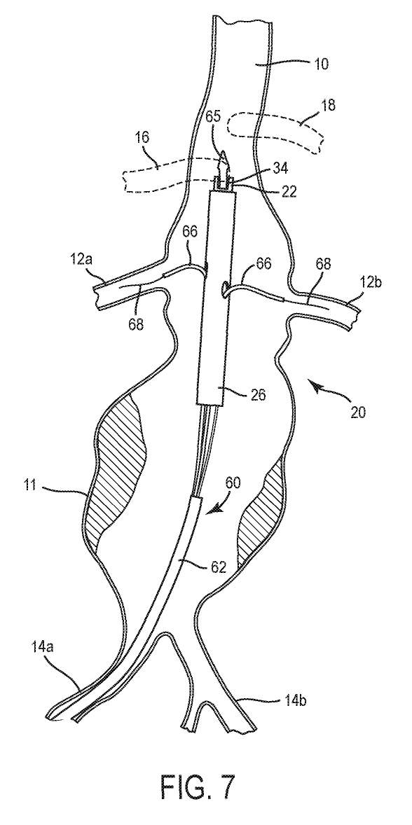

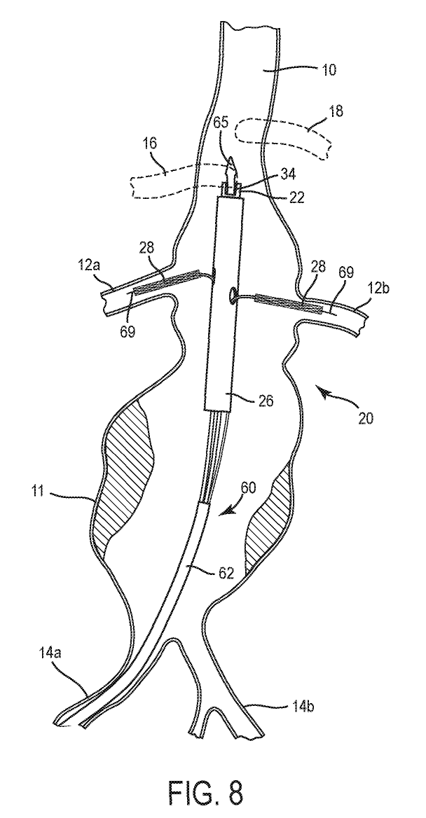

With reference to FIGS. 1, 6, and 7, in various embodiments a respective pre-curved angiographic catheter 66 is tracked over each pre-cannulated guidewire 64 from the central lumen 30 of the main graft body 22, through a corresponding fenestration 24a, 24b, and through a corresponding conduit 48 in the filling structure 26. In various embodiments, each pre-cannulated guidewire 64 is withdrawn and a corresponding guidewire 68 is advanced through the ostium of the target vessel to cannulate the target vessel. In various embodiments, each angiographic catheter 66 is advanced into the corresponding target vessel. With reference to FIGS. 7 and 8, in some embodiments, each guidewire 68 is withdrawn and a corresponding stiffer guidewire 69 is advanced into each target vessel.

In various embodiments, the angiographic catheter 66 is then withdrawn and each collapsed support structure 28 is advanced over the corresponding stiffer guidewire 69 into the corresponding target vessel. Each support structure 28 is then expanded in the corresponding target vessel. Each support structure 28 expands against the corresponding target vessel to secure the endoluminal prosthesis 20 in the desired position. As illustrated in FIGS. 8, 9, and 10, in some embodiments, each support structure 28 comprises a balloon expandable stent. In some embodiments, each support structure 28 comprises a self-expandable stent and is expanded with a proximal retraction of a corresponding outer sheath. In some embodiments, each support structure 28 may be any suitable structure for maintaining the patency of the target vessel and providing fixation for the endoluminal prosthesis 20.

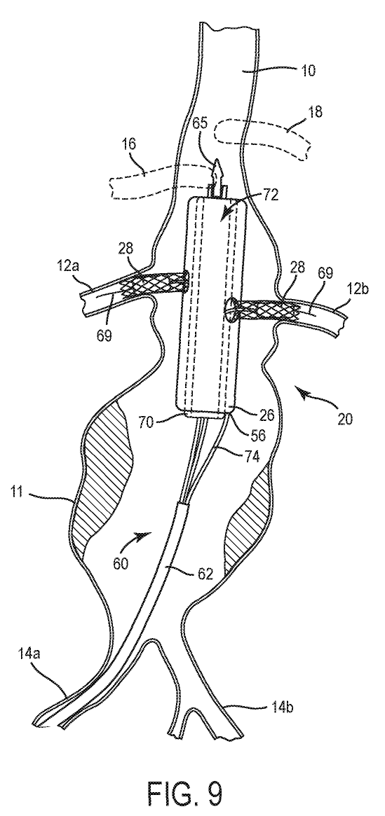

In various embodiments, with each support structure 28 expanded (e.g., through the inflation of a corresponding balloon catheter), the filling structure 26 is filled, as illustrated in FIG. 9. With reference to FIGS. 2 and 9, in various embodiments the inner lumen 46 is supported, such as with a balloon catheter 70 or other expansible structure that is inflated or expanded to open the inner lumen 46. In some embodiments, the balloon catheter 70 is formed from a generally compliant material, and has a maximum diameter or width which is at or slightly larger than a desired diameter or width of the inner lumen 46 through the deployed filling structure 26. In various embodiments, the balloon catheter 70 is configured to provide an open channel 72 through which blood can continue to flow throughout a filling procedure. By preventing total occlusion of the aorta 10, the forces applied to the endoluminal prosthesis 20 during deployment by blood flow can be minimized. In some embodiments, the filling structure 26 is first inflated with a temporary filling medium. For example, in an exemplary embodiment, the filling structure 26 is first filled with a saline solution. The temporary filling medium inflates the filling structure 26 and allows the positioning of the filling structure 26 and the seal with the aortic wall to be verified (e.g., with angiography).

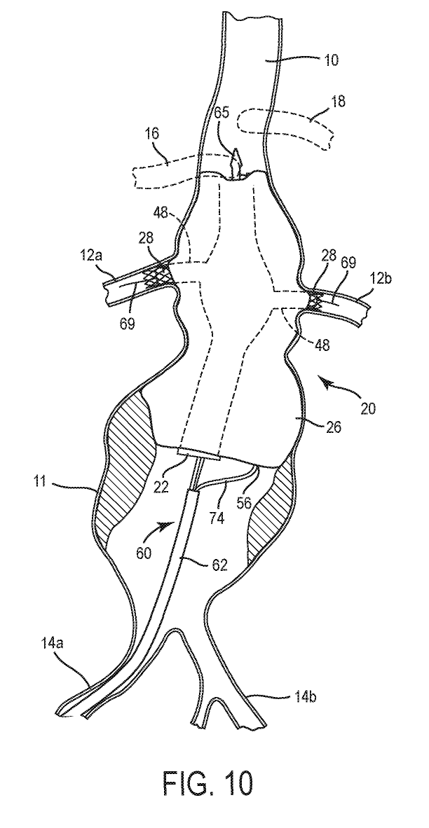

With reference to FIGS. 2 and 10 in various embodiments, after the filling structure 26 is properly positioned, the filling structure 26 is aspirated and a hardenable filling medium is introduced into the internal volume 40. In various embodiments, the hardenable filling medium is introduced into the internal volume 40 of the filling structure 26 with a filling tube 74 through an opening in a wall of the filling structure 26, such as through the valve 56. In some embodiments, the internal volume 40 is one continuous volume and the filling medium is introduced through a single valve 56. In some embodiments, as described in more detail below, the internal volume 40 is separated into multiple chambers or compartments and the filling structure 26 includes multiple filling valves to allow each of the multiple chambers or compartments to be individually filled with the filling medium.

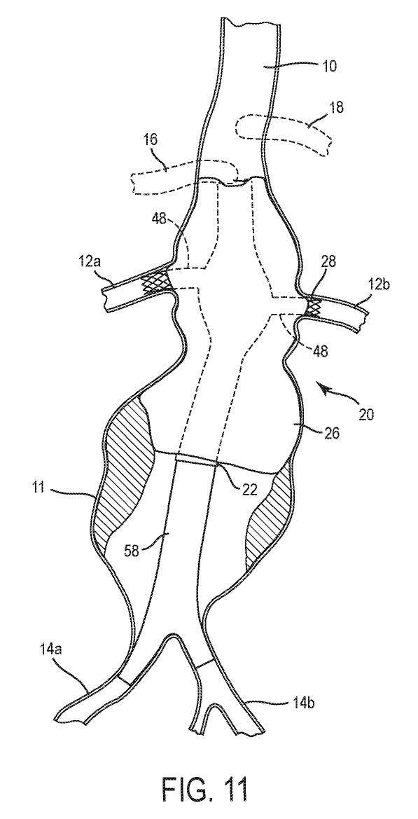

Filling of the internal volume 40 expands the outer wall 42 of the filling structure 26 outwardly so that it conforms to the inner surface of the aorta 10 and the aneurism 11, as illustrated in FIG. 11. With reference to FIGS. 2 and 11, in various embodiments each support structure 28 prevents the corresponding conduit 48 from collapsing and maintains an open passage from the inner lumen 46 to the corresponding renal artery 12a, 12b. In various embodiments, the outer wall 42 of the filling structure 26 forms a seal with the aortic wall surrounding the ostia of the renal arteries 12a, 12b.

In various embodiments, after the filling material has been introduced to the filling structure 26, the fluid filling material is cured or otherwise hardened to provide for the permanent implant having a generally fixed structure which will remain in place in the particular aneurismal geometry. In some embodiments, after the filling material has been cured, the seal between the filling structure 26 and the walls of the aorta 10 is verified (e.g., with angiography). In some embodiments, the balloon catheter 70 (refer to FIG. 9) supporting the inner lumen 46 of the filling structure 26 is aspirated, the balloon catheter expanding each support structure 28 is aspirated and removed, the lockwire is freed, and the deployment catheter 60 (refer to FIG. 9) is withdrawn. Methods for curing or hardening the filling material vary depending on the nature of the filling material. For example, certain exemplary polymers are cured by the application of energy, such as heat energy or ultraviolet light. Other exemplary polymers are cured when exposed to body temperature, oxygen, or other conditions which cause polymerization of the fluid filling material. Still other exemplary polymers are mixed immediately prior to use and simply cure after a fixed time, such as minutes.

Additionally, any of the endoluminal prostheses described herein can be used independently or can be used in conjunction with one or more additional grafts, including the grafts disclosed herein or any other suitable grafts such as other tubular or bifurcated grafts. For example, without limitation, the endoluminal prosthesis 20 can be used in conjunction with an additional prosthesis configured the same as or similar to the endoluminal prosthesis 20 to accommodate additional branch vessels, such as the celiac artery 18 or the SMA 16, and/or in conjunction with any other suitable prostheses, such as without limitation a bifurcated prosthesis, such as the second endoluminal prosthesis 58 illustrated in FIG. 11. Such additional prosthesis may be deployed in the same or similar procedure as the endoluminal prosthesis 20, and may be deployed prior to the deployment of the endoluminal prosthesis 20 or in a subsequent procedure to the deployment of the endoluminal prosthesis 20. For example, in some embodiments, the endoluminal prosthesis 20 is deployed in a secondary procedure to treat an aneurysm previously treated with a bifurcated prosthesis.

Some arrangements are directed to methods of deploying an endoluminal prosthesis, such as without limitation the endoluminal prosthesis 20 described above, including inserting a delivery catheter into an artery, deploying an endoluminal prosthesis in a desired location, providing a pre-cannulated guidewire proximate to a target branch artery, tracking a pre-curved angiographic catheter over the pre-cannulated guidewire from a central lumen of a main graft body through a fenestration or other opening in the main graft body and a filling structure, withdrawing the pre-cannulated guidewire and advancing a guidewire through the ostium of the target vessel to cannulate the target vessel, advancing an angiographic catheter into the target vessel, withdrawing the guidewire and advancing a stiffer guidewire into the target vessel, advancing a collapsed support structure over the guidewire into the target vessel, expanding the support structure in the target vessel, inflating a balloon to support a central lumen of the filling structure, pre-filling the filling structure with a temporary filling medium, verifying the seal and position of the filling structure through angiography, aspirating the temporary filling medium from the filling structure, filling the filling structure with a hardenable filling medium, and verifying the seal of the filling structure through angiography. In some embodiments, the method of deploying an endoluminal prosthesis further includes deploying a second endoluminal prosthesis in such a way that it interacts with the first endoluminal prosthesis, such as to form a continuous lumen through which blood may flow. The steps of the foregoing procedures can be performed in the sequence described, or can be performed in any suitable sequence. In some arrangements, the target branch vessels are the renal arteries. For example, in some embodiments, the step of filling the filling structure with the hardenable filling medium is performed after deploying a second endoluminal prosthesis into the artery.

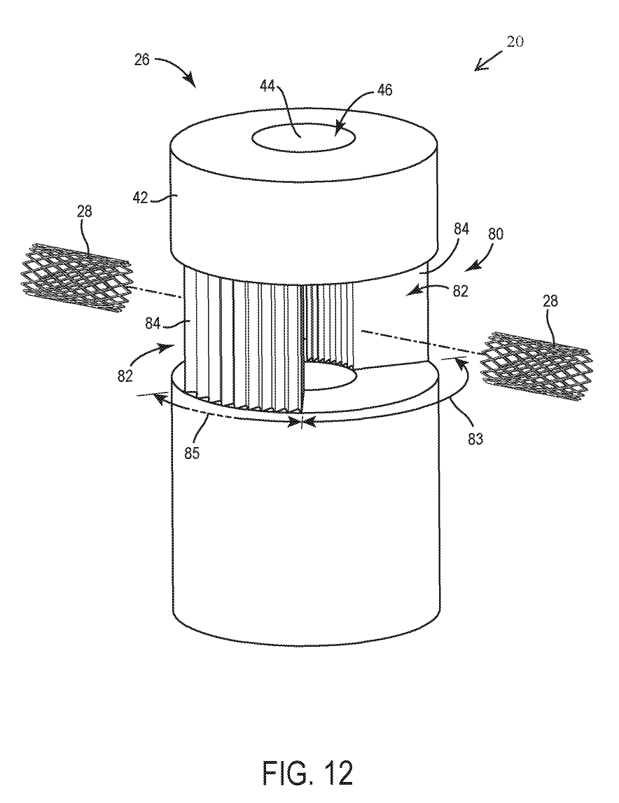

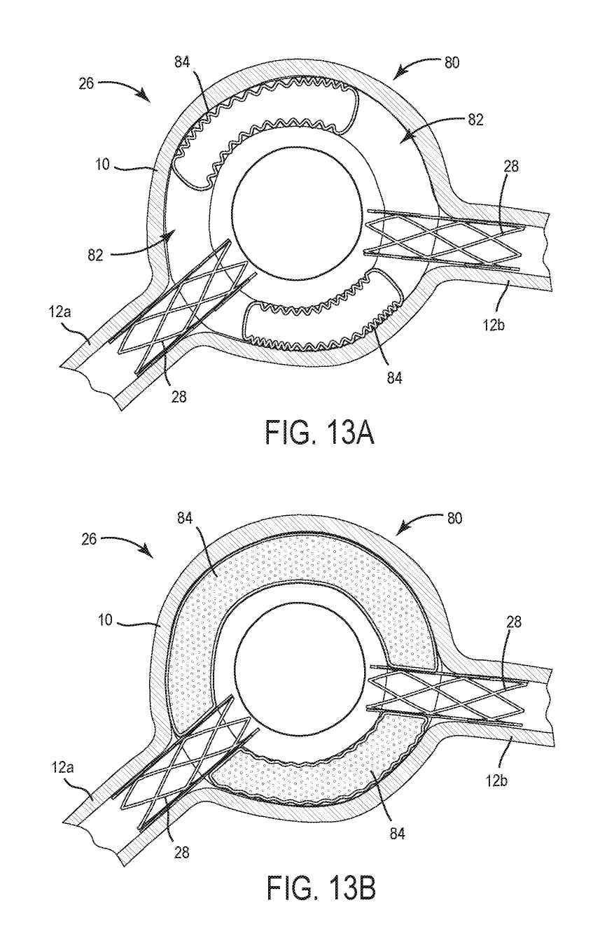

FIG. 12 is a perspective view of another embodiment of the endoluminal prosthesis 20. FIGS. 13A and 13B are cross-sectional views of the endoluminal prosthesis 20 of FIG. 12 deployed in a patient's anatomy. With reference to FIGS. 12 and 13A, in various embodiments the filling structure 26 includes a variable geometry portion 80 to accommodate a wide variety of positions for each support structure 28. In some embodiments, the variable geometry portion 80 defines a window 82 on both sides of the variable geometry portion 80 that each have an arc length 83 that is greater than a width of the corresponding support structure 28. In some embodiments, a variable length structure or shutter 84 is provided at the lateral edge on both sides of each window 82. In various embodiments, the shutters 84 are shaped to fill each window 82 and are oversized relative to each window 82. That is, in various embodiments, the shutters 84 are larger in at least one dimension than the corresponding dimension of each window 82 (e.g., circumference, arc length, height, etc.) to form a seal with the surfaces of the filling structure 26 defining the top and bottom of each window 82. In some embodiments, each shutter 84 has a compressed length 85 that can be expanded to a length equal to or greater than the compressed length plus the arc length 83 of the window 82 such that a single shutter 84 can seal an entire window 82.

In various embodiments, during deployment of the endoluminal prosthesis 20, the variable geometry portion 80 is positioned such that each window 82 is aligned with a corresponding target vessel. In some embodiments, each support structure 28 is advanced through the corresponding window 82 and into the corresponding target vessel and then expanded using a balloon catheter or other suitable expanding mechanism. In various embodiments, each shutter 84 is inflated by filling the shutter 84 with the filling medium. In some such embodiments, as each shutter 84 is filled, it expands (e.g., unfurls) to fill each window 82 up to a corresponding side of each support structure 28. In various embodiments, the oversized nature of each shutter 84 allows each window 82 to be filled on both sides of the corresponding support structure 28 in the window 82 regardless of the positioning of the corresponding support structure 28 in the window 82. In some embodiments, each shutter 84 is in fluid communication with the internal volume of the rest of the filling structure 26 is filled from a main filling valve for the filling structure 26. In some embodiments, each shutter 84 is fluidly isolated from the remainder of the filling structure 26 and is filled through one or more dedicated filling valves.

In various embodiments, the filling structure 26 including the variable geometry portion 80 is deployed at a desired position within the aorta 10 with the variable geometry portion 80 aligned with target branch vessels in which the support structures 28 will be deployed. In some embodiments, a pre-cannulated guidewire extends from a central lumen of a graft body, through a corresponding fenestration in the graft body, through the corresponding window 82 of the variable geometry portion 80, and out towards a nosecone of a deployment catheter. In some embodiments, additional radiopaque markers are provided on the outer wall 42 or the inner wall 44 of the filling structure 26 proximate each window 82 to aid in locating each window 82. In various embodiments, the deployment continues as described above, with each collapsed support structure 28 being advanced over a corresponding stiffer guidewire through the corresponding window 82 and into the corresponding target vessel. Each support structure 28 can then be expanded in the corresponding target vessel.

With reference to FIGS. 12 and 13B, in various embodiments the filling structure 26 is properly positioned, the filling structure 26 is prefilled with saline, aspirated, and then filled with a hardenable filling medium. In some embodiments, each shutter 84 is in fluid communication with the internal volume of the filling structure 26 such that each shutter 84 expands to fill each window 82 simultaneously with the expansion of the filling structure 26. In some embodiments, if each shutter 84 is fluidly isolated from the internal volume of the rest of the filling structure 26, the shutters 84 are separately filled with the hardenable filling medium through a separate filling valve. In various embodiments, each support structure 28 prevents each shutter 84 from entirely occluding the corresponding window 82 and maintains an open passage from the inner lumen 46 to the corresponding target vessel.

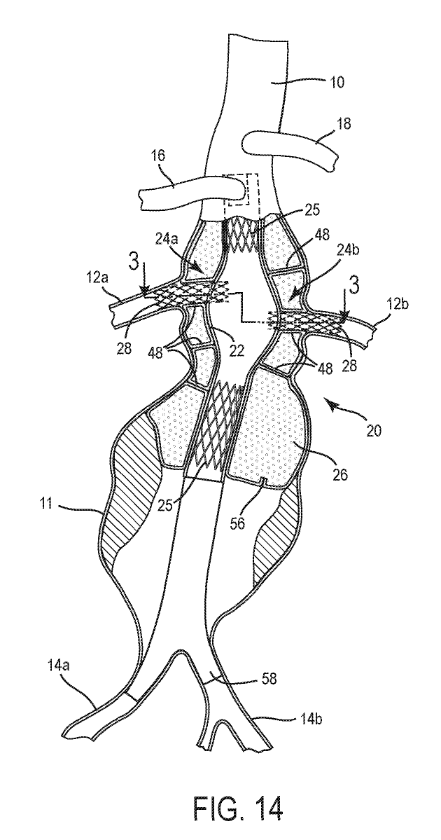

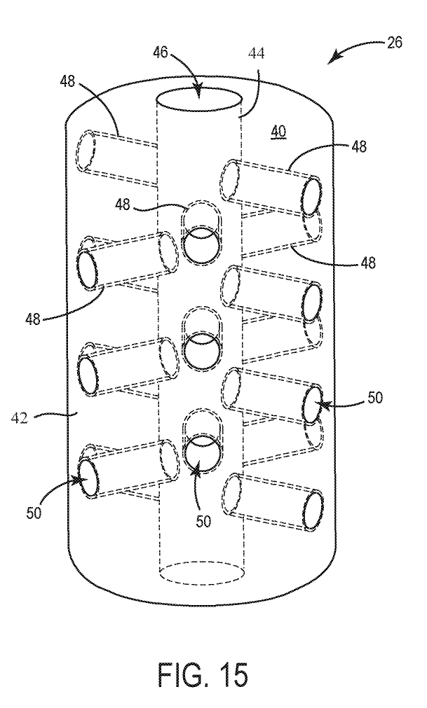

FIG. 14 is a partial section view of another embodiment of the endoluminal prosthesis 20 deployed in a patient's anatomy, including an embodiment of the filling structure 26 with a multitude of conduits 48. FIG. 15 is a perspective view of a schematic representation of the embodiment of the filling structure 26 with the multitude of conduits 48. With reference to FIGS. 14 and 15, in various embodiments the filling structure 26 includes the multitude of conduits 48, such as more than two, positioned along a length of the filling structure 26 and extending in a variety of directions. In various embodiments, the multitude of conduits 48 allow the filling structure 26 to be used with a variety of patients without having to have the filling structure 26 be chosen or fabricated to match a particular patient geometry being treated.

In various embodiments, when deployed, the endoluminal prosthesis 20 is equipped with a number of pre-cannulated guidewires corresponding to the number of vessels to be cannulated. The pre-cannulated guidewires are provided in the approximate locations of the target vessels (e.g., the renal arteries 12a, 12b, the SMA 16, the celiac artery 18, etc.). In various embodiments, each support structure 28 is deployed through the corresponding conduit 48 aligned with a corresponding target vessel.

In various embodiments, the filling structure 26 is properly positioned, the filling structure 26 is prefilled with saline, aspirated, and then filled with a hardenable filling medium. In various embodiments, filling of the internal volume 40 of the filling structure 26 expands the outer wall 42 of the filling structure 26 outwardly so that it conforms to the inner surface of the aorta 10 and the aneurismal space. In various embodiments, each support structure 28 prevents a corresponding conduit 48 aligned with a target vessel from collapsing and maintains open a passage from the inner lumen 46 to the target vessel. Meanwhile, in various embodiments, the filling material or medium collapses any conduits 48 in which support structures 28 are not disposed, resulting in a permanent implant.

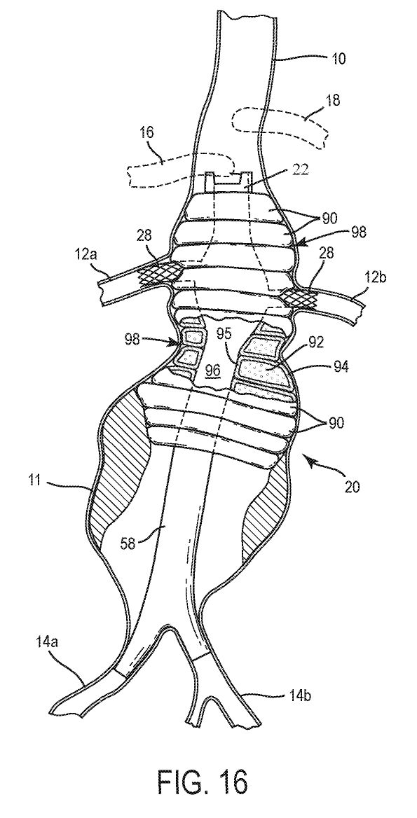

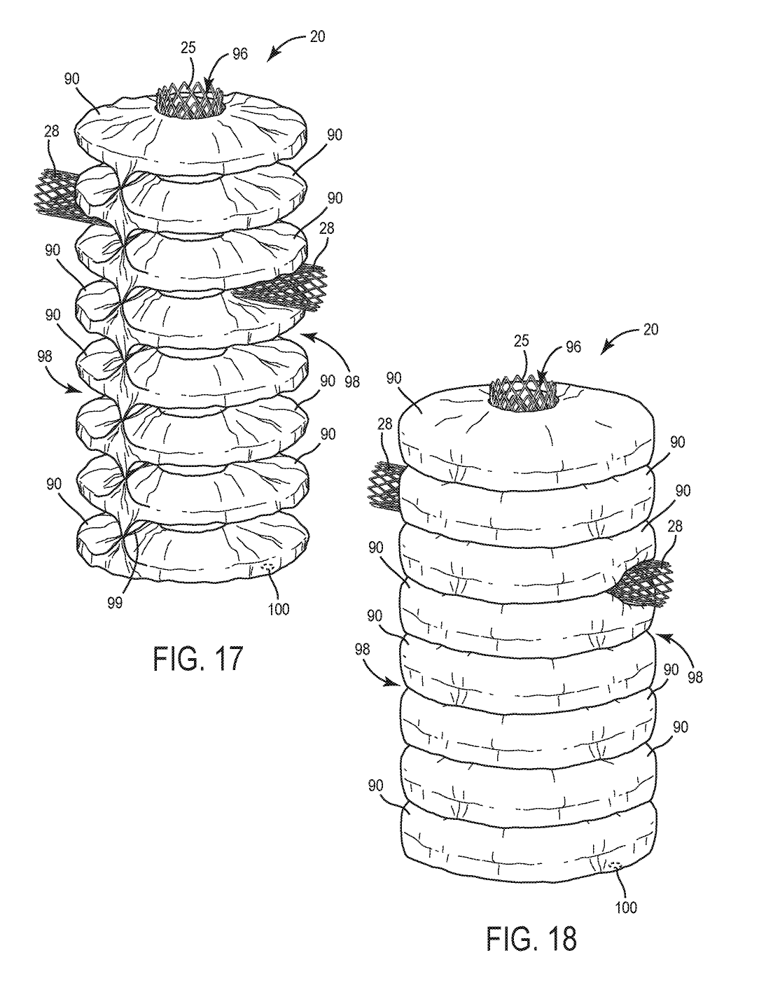

FIG. 16 is a partial section view of another embodiment of the endoluminal prosthesis 20 deployed in a patient's anatomy including a multitude of horizontal filling structures 90. FIG. 17 is a perspective view of support structures 28 extending between horizontal filling structures 90 in an uninflated state. FIG. 18 is a perspective view of support structures 28 extending between horizontal filling structures 90 in an inflated state. With reference to FIGS. 16, 17, and 18, in various embodiments, the horizontal filling structures 90 are generally donut-shaped bodies that include an internal volume 92 defined by an outer wall 94 and inner wall 95. In various embodiments, the stack of horizontal filling structures 90 defines an inner lumen 96. In some embodiments, the inner lumen 96 is configured to receive the main graft body 22. In various embodiments, a space or gap 98 is provided between adjacent horizontal filling structures 90. In some such embodiments, the gaps 98 between the horizontal filling structures 90 provide multiple passages for a support structure 28 to pass through, and the stack of horizontal filling structures 90 is positioned relative to the main graft body 22 such that a gap 98 is aligned with a corresponding fenestration of the main graft body 22. While only two support structures 28 are illustrated in FIG. 16 disposed in the renal arteries 12a and 12b, respectively, the multitude of gaps 98 provided between the horizontal filling structures 90 are able to accommodate a multitude of support structures. For example, in other embodiments, the gaps 98 between the horizontal filling structures 90 receive support structures 28 disposed in other branch vessels, such as the SMA 16 and the celiac artery 18.

With reference to FIG. 17, in some embodiments, the horizontal filling structures 90 are coupled together with attachment portions 99. As illustrated in FIG. 17, in some embodiments the horizontal filling structures 90 are joined directly to each other with at least one attachment portion 99. In various embodiments, the horizontal filling structures 90 are joined together with a web or other separate body coupled between horizontal filling structures 90.

With reference to FIGS. 16, 17, and 18, in various embodiments the horizontal filling structures 90 includes at least one valve 100 to permit the introduction of the filling material or medium into the internal volume 92. In some embodiments, the internal volumes 92 of the horizontal filling structures 90 are in fluid communication with each other and are filled from a single filling valve 100. In some embodiments, the internal volumes 92 of the horizontal filling structures 90 are fluidly isolated from each other and are each filled through dedicated filling valves.

In various embodiments, during deployment of the endoluminal prosthesis 20 the horizontal filling structures 90 are positioned such that one of the gaps 98 is aligned with a target vessel. In some such embodiments, the corresponding support structure 28 for the target vessel is advanced through the gap 98 and into the target vessel and then expanded using a balloon catheter or other suitable expanding mechanism. The horizontal filling structures 90 are inflated by filling the horizontal filling structures 90 with the filling medium. In various embodiments, upon inflation with a filling material or medium delivered into the internal space 92, the outer wall 94 expands outwardly, upwardly and downwardly, and the inner wall 95 expands inwardly. In various embodiments, the inward expansion contracts the inner lumen 96, forming a seal with the main graft body 22. In various embodiments, the outward expansion expands the outer diameter of the horizontal filling structures 90, allowing the horizontal filling structures 90 to match the particular patient geometry being treated. The upward and downward expansion closes the gaps 98 between adjacent horizontal filling structures 90 and forms a seal around the support structures 28 extending through the gaps 98.

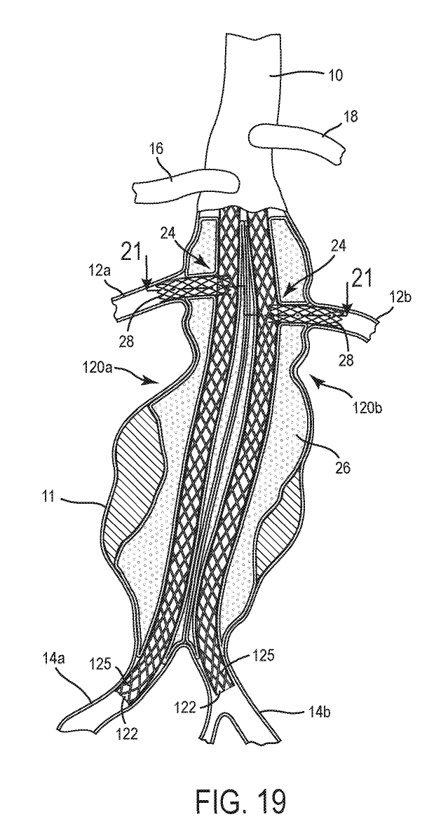

FIG. 19 is a partial section view of a patient's vasculature illustrating a pair of endoluminal prostheses 120a, 120b in accordance with an embodiment deployed in a desired position within the patient's aorta 10. For reference, also illustrated are the patient's first and second renal arteries 12a, 12b, respectively, the patient's first and second iliac arteries 14a, 14b, respectively, the patient's superior mesenteric artery (SMA) 16, and the patient's celiac artery 18. An infrarenal abdominal aortic aneurysm 11 is also shown between the renal arteries 12a and 12b and the iliac arteries 14a and 14b and may have regions of mural thrombus over portions of its inner surface.

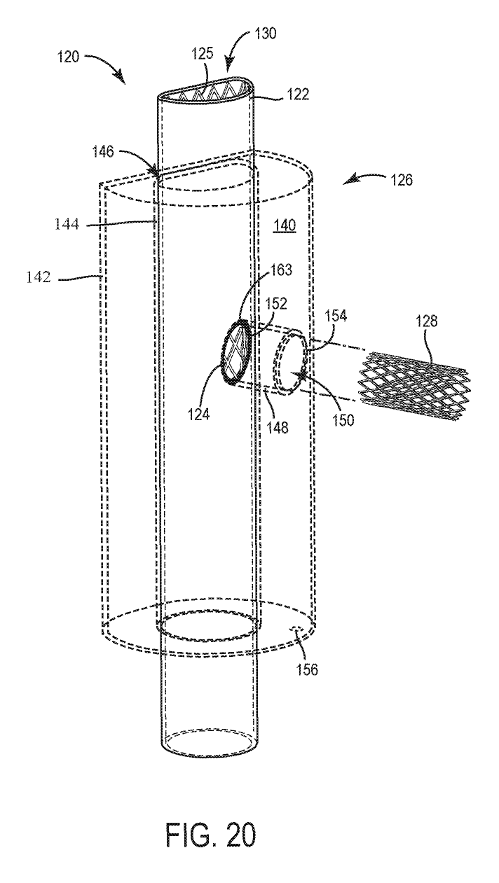

FIG. 20 is a front elevation view of an endoluminal prostheses 120 in accordance with an embodiment that can be a design for each of the pair of endoluminal prostheses 120a, 120b illustrated in FIG. 19. With reference to FIGS. 19 and 20, the first endoluminal prosthesis 120a is deployed on the one side of the aorta 10 to facilitate blood flow to the first renal artery 12a and to the first iliac artery 14a and the second endoluminal prosthesis 120b is deployed on the other side of the aorta 10 to facilitate blood flow to the second renal artery 12b and to the second iliac artery 14b. In various embodiments, the endoluminal prostheses 120a, 120b generally have asymmetric configurations which are configured to be positioned adjacent to each other within the aneurism 11 and to, in combination, fill that space. Because the first and second endoluminal prostheses 120a, 120b are generally similar in construction, the same reference numerals will be used to describe the respective components of the first and second endoluminal prostheses 120a, 120b.

In various embodiments, the endoluminal prosthesis 120 includes a main graft body 122 including a fenestration 124 (e.g., scallop, cutout, opening, etc.), a support structure 125, a double-walled filling structure 126 in the area of the aneurysm 11, a support structure 128 extending through the fenestration 124 and through the filling structure 126.

The main graft body 122 defines a central lumen 130. The main graft body 122 provides a synthetic vessel wall that channels the flow of blood through the diseased portion of the blood vessel. In various embodiments, the endoluminal prosthesis 120 is positioned with the fenestration 124 aligned with a branch of the aorta 10. In some embodiments, the fenestration 124 is aligned with one of the renal arteries 12a, 12b such that a fluid path is formed from the central lumen 130 to the corresponding renal artery 12a, 12b. The main graft body 122 can be formed from any suitable material, such as, but not limited to, ePTFE, PTFE, and paralyne.

In some embodiments, the support structure 125 (e.g., a covered stent, a bare wire stent, etc.) or any other suitable stent or anchoring device is deployed within the central lumen 130 of the main graft body 122 to secure the main graft body 122 in the desired location. In various embodiments, the support structure 125 compresses the main graft body 122 against the fillable structure 126 provided between the main graft body 122 and the wall of the blood vessel and secures the main graft body 122 and the fenestration 124 in the desired locations. In some embodiments, the support structure 125 is formed from any number of resilient metals such as stainless steel, cobalt-chromium (CoCr), nitinol or resilient polymers. The support structure 125 may be coupled to the inside or outside surface of the main graft body 122. In various embodiments, the fenestration 124 extends through the support structure 125, such that the support structure 128 is able to pass through the support structure 125.

In some embodiments, the support structure 125 extends the entire length of the main graft body 122. In some embodiments, the support structure 125 only extends along a portion of the main graft body 122. For example, the support structure 125 can include an upper portion disposed at the top of the endoluminal prosthesis 120, proximate to the renal arteries 12a, 12b, and a lower portion separate from the upper portion and disposed at the bottom of the endoluminal prosthesis 120, proximate to the iliac arteries 14a, 14b.

In some embodiments, the main graft body 122 and support structure 125 can have any of the features of the structures disclosed in U.S. patent application Ser. No. 12/478,208, filed on Jun. 4, 2009 (titled “Docking Apparatus and Methods of Use”), which is hereby incorporated by reference in its entirety as if fully set forth herein.

In various embodiments, the filling structure 126 surrounds the main graft body 122 and, when inflated, occupies the annular space between the main graft body 122 and the walls of the aorta 10. In some embodiments, the filling structure 126 includes an internal volume 140 defined between an outer wall 142 and inner wall 144. In various embodiments, the geometry of the filling structure 126 is chosen or fabricated to match the particular patient geometry being treated. In various embodiments, upon inflation with a filling material or medium delivered into the internal space 140, the outer wall 142 of the filling structure 126 expands radially outwardly and the inner wall 144 expands inwardly. In some embodiments, the inner wall 144 defines an inner lumen 146 that is configured to receive the main graft body 122.

In various embodiments, the filling structure 126 further includes at least one conduit 148 that extends through the internal volume 140 of the filling structure 126 and is aligned with the fenestration 124 of the main graft body 122. The conduit 148 defines a passage 150 that is separate from the internal volume 140. The passage 150 is open on a first end 152 into the inner lumen 146 and is open on a second end 154 into the space surrounding the filling structure 126. In some embodiments, the filling structure 126 is coupled to the main graft body 122 and/or the support structure 125 with sutures to maintain a desired alignment of the conduit 148 with respect to the fenestration 124. In some embodiments, the filling structure 126 is coupled to the main graft body 122 and/or the support structure 125 with an adhesive or other suitable attachment mechanism. In some embodiments, the filling structure 126 is integrally formed with the main graft body 122 such that the conduit 148 is aligned with the fenestration 124.

In various embodiments, the filling structure 126 includes at least one valve 156 to permit the introduction of the filling material or medium into the internal volume 140 of the filling structure 126. In some embodiments, the valve 156 comprises a simple flap valve. Other more complex ball valves, and other one-way valve structures may be used for the valve 156. In some embodiments, the valve 156 comprises a two-way valve structure to permit both filling and selective emptying of the internal volume 140 of the filling structure 126. In some embodiments, a filling tube includes a needle or other filling structure to pass through the valve 156 to permit both filling and removal of filling medium.

In some embodiments, the main graft body 22 and/or the support structure 125 extends beyond the lower end of the filling structure 126 and passes through an aneurysmal region within the iliac artery 14a, 14b, thus allowing the structure to treat the iliac aneurysm as well as the aortic aneurysm.

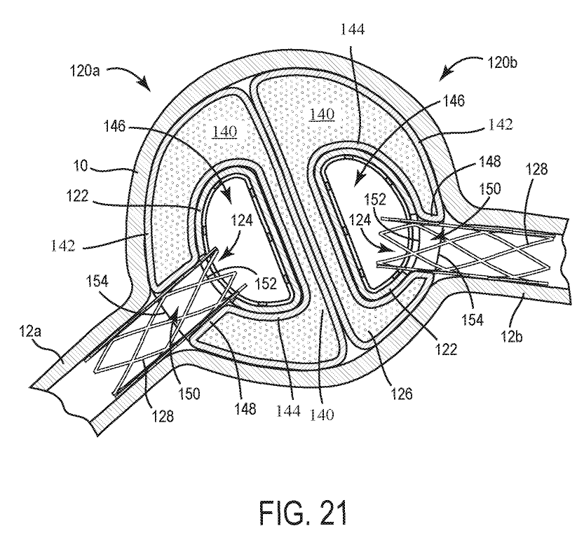

FIG. 21 is a cross-sectional view of the embodiment of the endoluminal prostheses 120a, 120b deployed in the patient's anatomy, taken through line 21-21 in FIG. 19. With reference to FIGS. 19, 20, and 21, in some embodiments the upper ends of the support structures 125 are formed to have D-shaped cross-sections when expanded. In some such embodiments, the flat face of each support structure 125 faces the flat face of the other support structure 125, with the remaining portions (e.g., the curved faces) of the support structures 125 facing the inner wall of the aorta 10. In this way, most of the cross-sectional area of the aorta 10 will be covered with the support structures 125, thus enhancing blood flow through the endoluminal prostheses 120a, 120b. In some embodiments, a clip or other fastening device, link, or tether, is provided to connect the upper ends of the support structures 125. By attaching the ends of the support structures 125, the ends of the endoluminal prostheses 120a, 120b are stabilized and the risk of support structure migration is reduced.

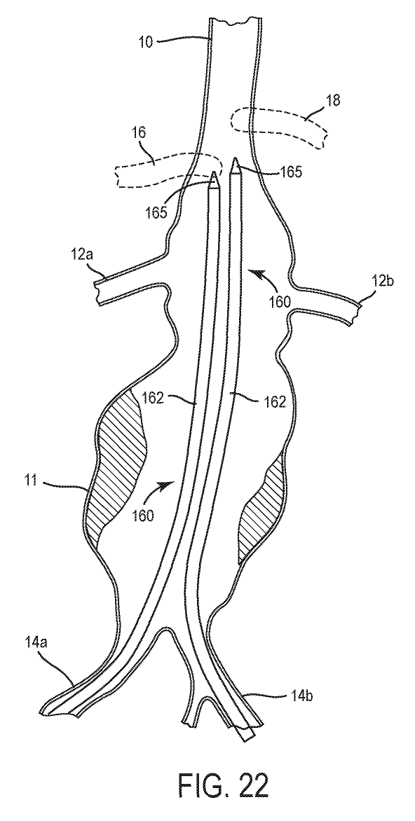

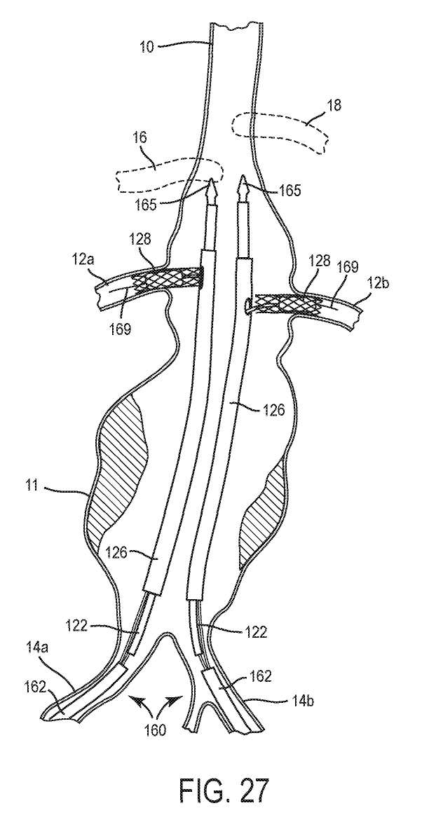

FIGS. 22, 23, 24, 25, 26, 27, 28, and 29 are schematic representations in accordance with an embodiment a process of positioning and implanting the endoluminal prostheses 120a, 120b (refer to FIG. 19) in a desired aortic location. In various embodiments, a pair of deployment catheters 160 are positioned in the aorta 10, as illustrated in FIG. 22. With reference to FIGS. 19 and 22, the deployment catheters 160 include hollow outer sheaths 162 in which the endoluminal prostheses 120a, 120b are compressed and loaded for delivery to a desired location. According to an exemplary embodiment, a deployment catheter 160 is advanced into the aorta 10 from each of the iliac arteries 14a, 14b. According to an exemplary embodiment, the deployment catheters 160 are each advanced over a guidewire through a puncture in the patient's groin accessing the corresponding iliac artery 14a, 14b by the Seldinger technique. In various embodiments, the deployment catheters 160 are positioned to deploy the endoluminal prostheses 120a, 120b in such a way as to maintain patency of the branch blood vessels.

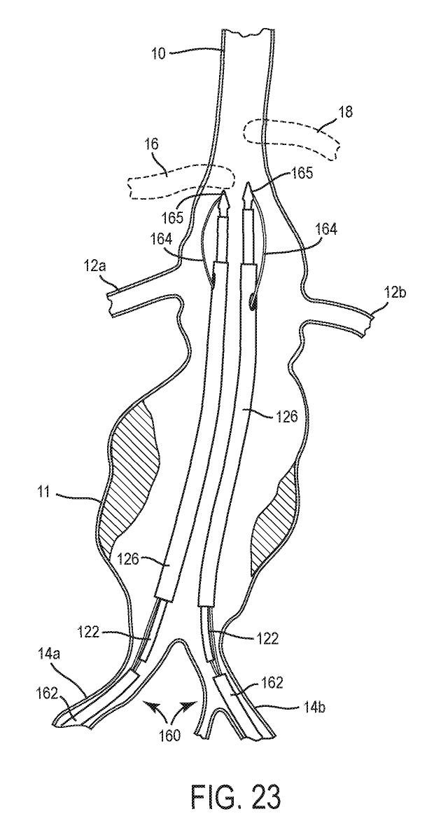

In some embodiments, a position and angular orientation (e.g., rotation) of the endoluminal prostheses 120a, 120b are controlled such that the fenestrations 124 are aligned with the target vessels (e.g., the renal arteries 14a, 14b). The two endoluminal prostheses 120a, 120b are able to be repositioned independently from each other, allowing a greater flexibility in the positioning of the endoluminal prostheses 120a, 120b without occluding the branch arteries. In various embodiments, once properly positioned within the aorta 10 the outer sheaths 162 of the deployment catheters 160 are retracted to expose the filling structures 126, as illustrated in FIG. 23. With reference to FIGS. 19, 20, and 23, in various embodiments the endoluminal prostheses 120a, 120b are fixed in place during the procedure, such as with lockwires. In some embodiments, one or more pre-cannulated guidewires 164 are provided. In various embodiments, each pre-cannulated guidewire 164 extends from the central lumen 130 of the corresponding main graft body 122, through the corresponding fenestration 124 in the main graft body 122, through the corresponding conduit 148 in the filling structures 126, and out towards a corresponding nosecone 165 of the corresponding deployment catheter 160. In various embodiments, the pre-cannulated guidewires 164 run under the support structures 128, which are stored in a collapsed state within the sheaths 162. In some embodiments, one or more radiopaque markers 163 are provided at the fenestrations 124 to facilitate the positioning of the pre-cannulated guidewires 164. In some embodiments, additional radiopaque markers are provided on the outer walls 142 and the inner walls 144 of the filling structures 126 proximate the conduits 148.

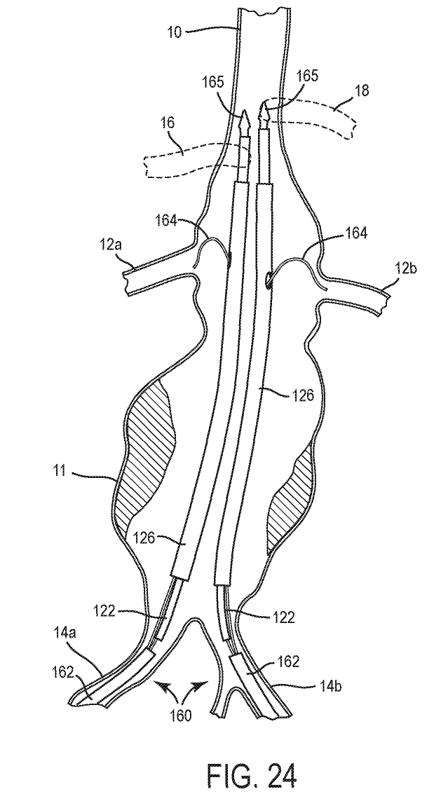

In some embodiments, two pre-cannulated guidewires 164 are provided in the approximate locations of the renal arteries 12a, 12b, as illustrated in FIG. 24. In some embodiments, three or more pre-cannulated guidewires are provided to accommodate additional vessels, such as the celiac artery 18 and/or the SMA 16 if the endoluminal prostheses are configured with support structures for additional vessels.

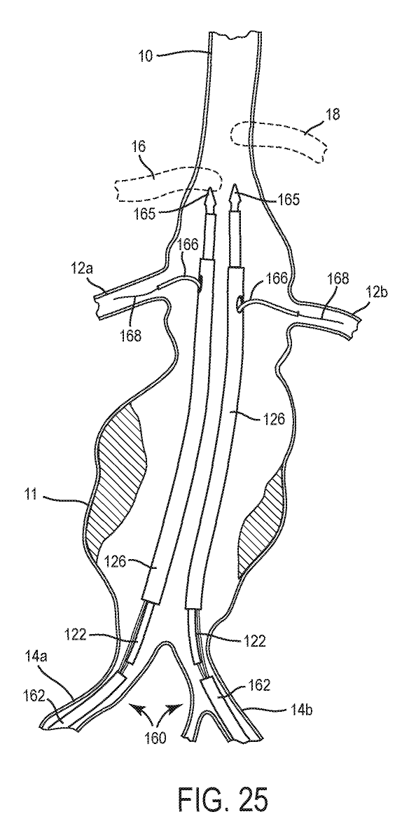

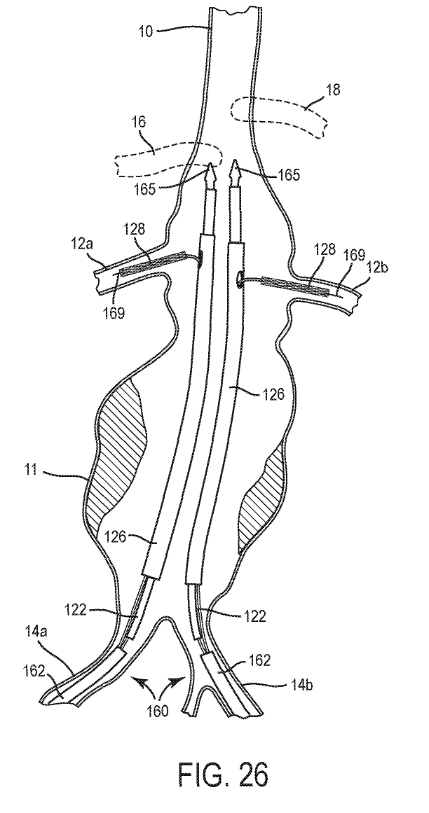

With reference to FIGS. 19, 20, 24, and 25, in some embodiments pre-curved angiographic catheters 166 are tracked over the pre-cannulated guidewires 164 from the central lumens 130 of the main graft bodies 122, through the fenestrations 124 and through the conduits 148 in the filling structures 126 (refer to FIG. 25). In various embodiments, the pre-cannulated guidewires 164 are withdrawn and guidewires 168 are advanced through the ostia of the target vessels to cannulate the target vessels. In some embodiments, the angiographic catheters 166 are advanced into the target vessels. In some embodiments, the guidewires 168 are withdrawn and stiffer guidewires 169 (refer to FIG. 26) are advanced into the target vessels.

With reference to FIGS. 25 and 26, in various embodiments the angiographic catheters 166 are then withdrawn and the collapsed support structures 128 are advanced over the stiffer guidewires 129 into the target vessels. The support structures 128 are then expanded in the target vessels. The support structures 128 expand against the target vessels to secure the support structures 128 in the desired positions. As illustrated in FIGS. 26, 27, and 28, in some embodiments, the support structures 128 are balloon expandable stents. In some embodiments, the support structures 128 are self-expandable stents and are expanded with the proximal retraction of an outer sheath. In some embodiments, the support structures 128 may be any suitable structures for maintaining the patency of the target vessels and providing fixation.

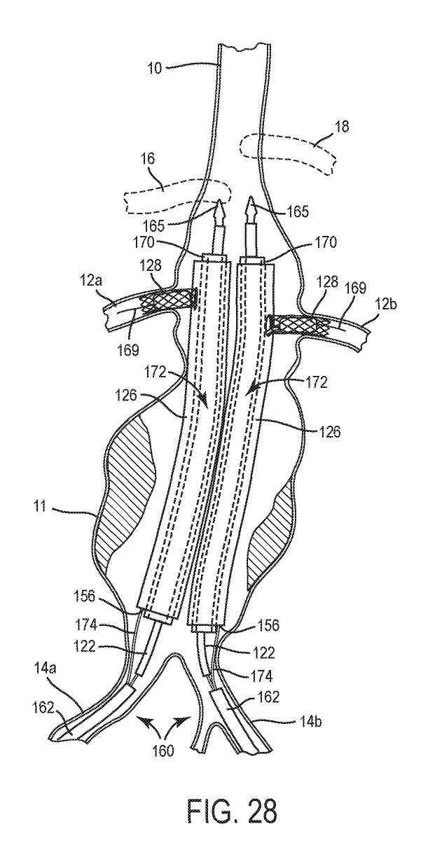

In various embodiments, with the support structures 128 expanded (e.g., through the inflation of balloon catheters), the filling structures 126 are filled, as illustrated in FIG. 28. With reference to FIGS. 20 and 28, in various embodiments the inner lumens 146 are first supported, such as with a balloon catheters 170 or other expansible structures that are inflated or expanded to open the inner lumens 146. In some embodiments, the balloon catheters 170 are formed from a generally compliant material, and have a maximum diameter or width which is at or slightly larger than the desired diameters or widths of the inner lumens 146 through the deployed filling structures 126. In some embodiments, the balloon catheters 170 are configured to provide open channels 172 through which blood can continue to flow throughout the filling procedure. By preventing total occlusion of the aorta 10, the forces applied to the endoluminal prostheses during deployment can be minimized. In some embodiments, the filling structures 126 are inflated with a temporary filling medium, such as a saline solution. In some such embodiments, the temporary filling medium inflates the filling structures 126 and allow the positioning of the filling structures 126 and the seal with the aortic wall to be verified (e.g., with angiography).

In some embodiments, one of the filling structures 126 and associated balloon catheters 170 is expanded first, followed by the other filling structure 126 and balloon catheter 170. In various embodiments, each filling structure 126 is inflated to fill generally half of the aneurismal volume. In some embodiments, after one filling structure 126 has been filled, the other filling structure 126 may be filled. In some embodiments, the two filling structures 126 are filled simultaneously.

In various embodiments, after the filling structures 126 are properly positioned, the filling structures 126 are aspirated and a hardenable filling medium is introduced into the internal volumes 140. In some embodiments, the hardenable filling medium is introduced into the internal volumes 140 with filling tubes 174 through openings in the walls of the filling structures 126, such as through the filling valves 156.

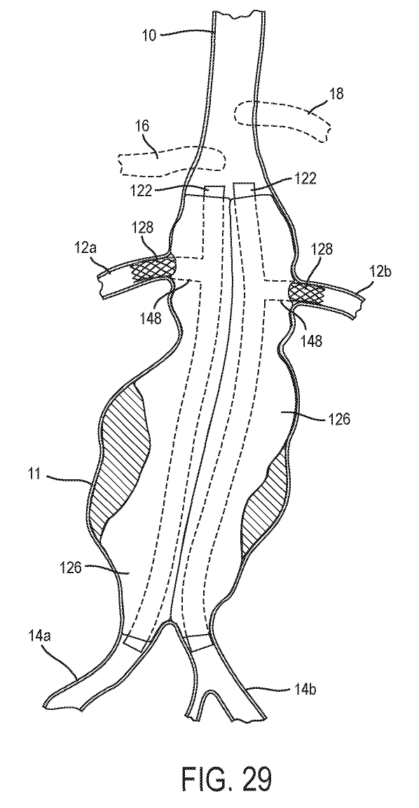

With reference to FIGS. 20 and 29, in various embodiments, the filling of the internal volumes 140 of the filling structures 126 expands the outer walls 142 of the filling structures 126 outwardly so that they conform to the inner surface of the aorta 10 and the aneurismal space. In various embodiments, the support structures 128 prevent the conduits 148 from collapsing and maintain open passages from the inner lumens 146 to the renal arteries 12a, 12b. The outer walls 142 of the filling structures 126 form a seal with the aortic wall surrounding the ostia of the renal arteries 12a, 12b.

In various embodiments, after the filling material has been introduced to the filling structures 126, the fluid filling material is cured or otherwise hardened to provide for a permanent implant having a generally fixed structure which will remain in place in the particular aneurismal geometry. In some embodiments, after the filling material has been cured, the seal between the filling structures 126 and the walls of the aorta 10 are verified (e.g., with angiography). In some embodiments, the balloon catheters 170 (refer to FIG. 28) in the inner lumens 146 of the filling structures 126 are aspirated, the balloon catheters that expanded the support structures 128 are aspirated and removed, the lockwires are freed, and the deployment catheters 160 (refer to FIG. 28) are withdrawn. Methods for curing or hardening the filling material depend on the nature of the filling material. For example, certain exemplary polymers are cured by the application of energy, such as heat energy or ultraviolet light. Other exemplary polymers are cured when exposed to body temperature, oxygen, or other conditions which cause polymerization of the fluid filling material. Still other exemplary polymers are mixed immediately prior to use and simply cure after a fixed time, such as minutes.

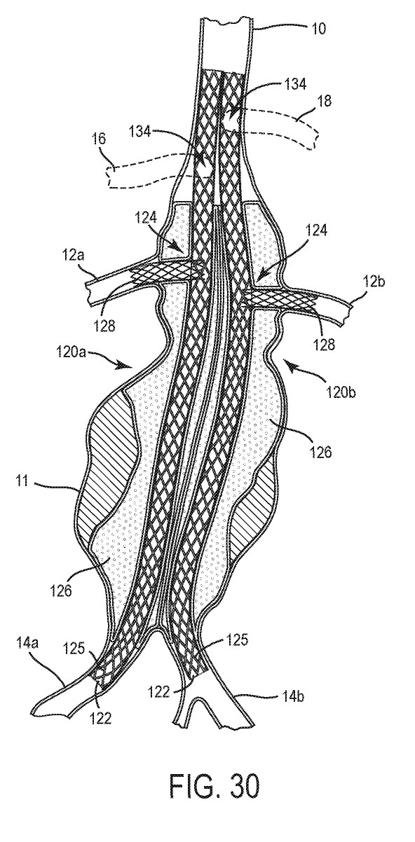

FIG. 30 is a front elevation view of another embodiment of the endoluminal prostheses 120a, 120b. In various embodiments, each of the endoluminal prostheses 120a, 120b includes a main graft body 122 including a fenestration 124 (e.g., scallop, cutout, opening, etc.), a support structure 125, a double-walled filling structure 126 in the area of the aneurysm 11, and support structures 128 that each extend through the respective fenestration 124 and through the respective filling structure 126.

In various embodiments, each of the endoluminal prostheses 120a, 120b further includes a respective second fenestration 134. In various embodiments, the second fenestration 134 is positioned such that, when the corresponding fenestration 124 is aligned with a first branch vessel, such as one of the renal arteries 12a, 12b, the second fenestration 134 is aligned with another branch vessel, such as the SMA 16, the celiac artery 18, etc. In various embodiments, additional conduits are provided in the filling structures 126 that are aligned with the second fenestrations 134. In some embodiments, additional support structures are deployed into the branch vessels aligned with the second fenestrations 134 in accordance with the process described above with respect to the fenestrations.

In some embodiments, the endoluminal prostheses 120a, 120b are deployed without the use of supports structures, such as support structure 125 or support structure 128. Instead, in some embodiments, the endoluminal prostheses 120a, 120b are positioned and fixed in place using only the interface between the vessel walls and the filling structures 126. According to an exemplary embodiment, the hardenable filling medium with which the internal volume of each filling structure 126 is inflated is a resilient material. In some embodiments, forces applied to the filling structures 126, such as the fluid pressure applied to the filling structures 126 by blood flow, cause the filling structures 126 to temporarily deform such that the forces are absorbed by the resilient filling medium and transferred to the vessel walls.

Some arrangements disclosed herein are directed to systems and methods of deploying an endoluminal prosthesis, such as, without limitation, the prostheses described above, including inserting a delivery catheter such as deployment catheter into an artery, deploying endoluminal prostheses in a desired location, providing pre-cannulated guidewires proximate to target branch arteries, tracking pre-curved angiographic catheters over the pre-cannulated guidewires from central lumen of main graft bodies through fenestrations or other openings in the main graft bodies and the filling structures, withdrawing the pre-cannulated guidewires and advancing guidewires through the ostia of the target vessels to cannulate the target vessels, advancing angiographic catheters into the target vessels, withdrawing the guidewires and advancing stiffer guidewires into the target vessels, advancing collapsed support structures over the guidewires into the target vessels, expanding the support structures in the target vessels, inflating balloons to support central lumens of the filling structures, pre-filling the filling structures with a temporary filling medium, verifying the seal and position of the filling structures through angiography, aspirating the temporary filling medium from the filling structures, filling the filling structures with a hardenable filling medium, and verifying the seals of the filling structures through angiography. The steps of the foregoing procedure in accordance with various embodiments can be performed in the sequence described, or can be performed in any suitable sequence, and one or more of the steps may be omitted in various embodiments. In some arrangements, the target branch vessels are the renal arteries. In some embodiments, the step of filling the filling structure with the hardenable filling medium is performed with one endoluminal prosthesis before the step of filling the filling structure with the hardenable filling medium is performed with a second endoluminal prosthesis.

The order or sequence of any process or method steps may be varied or re-sequenced according to alternative embodiments. Other substitutions, modifications, changes and omissions may also be made in the design, operating conditions, and arrangement of the various exemplary embodiments without departing from the scope of the present invention.

The construction and arrangement of the elements of the endoluminal prostheses as shown in the exemplary embodiments are illustrative only. Although embodiments of the present disclosure have been described in detail, those skilled in the art who review this disclosure will readily appreciate that many modifications are possible (e.g., variations in sizes, dimensions, structures, shapes, and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements. Some like components have been described in the present disclosure using the same reference numerals in different figures. This should not be construed as an implication that these components are identical in all embodiments; various modifications may be made in various different embodiments. It should be noted that the elements and/or assemblies of the enclosure may be constructed from any of a wide variety of materials that provide sufficient strength or durability, in any of a wide variety of colors, textures, and combinations.

What is claimed is:

1. A system, comprising:
a graft body with a fenestration in a side surface through which a support structure is insertable; and
a filling structure defining an internal volume that is fillable with a filling medium and configured with a conduit through the internal volume through which the support structure is insertable.

2. The system of claim 1,
wherein the conduit in the filling structure is alignable with the fenestration in the graft body such that the support structure is insertable through both the conduit in the filling structure and the fenestration in the graft body.

3. The system of claim 1,
wherein the fenestration in the graft body and the conduit in the filling structure are alignable with a renal artery.

4. The system of claim 1, further comprising:
a stent attached to the graft body at a portion of the graft body that is entirely above a location of the fenestration in the graft body.

5. The system of claim 1,
wherein the graft body is configured to have an enlarged portion having slack graft material to allow for the fenestration to be moved to align the fenestration with an artery.

6. The system of claim 1,
wherein the filling structure surrounds at least a portion of the graft body.

7. The system of claim 1,
wherein the filling structure is configured such that the conduit in the filling structure is an opening with a shutter that is closable around the support structure after the support structure has been inserted into the opening.

8. The system of claim 1,
wherein the filling structure is configured with a plurality of conduits positioned along a length of the filling structure.

9. The system of claim 8,
wherein the filling structure is configured such that each of the plurality of conduits is collapsible when the filling structure is filled with the filling medium.

10. A system, comprising:
a plurality of filling structures coupled together and including one or more gaps between at least two of the plurality of filling structures through which a support structure is insertable.

11. The system of claim 10,
wherein the plurality of filling structures are configured such that the one or more gaps are collapsible when the plurality of filling structures are filled with a filling medium.

12. The system of claim 10,
wherein the one or more gaps are alignable with renal arteries of a patient.
